(19) United States
(12) Patent Application Publication
(10) Pub. No.: US 2018/0052963 A1
(43) Pub. Date: Feb. 22, 2018

(54) ULTRASOUND DIAGNOSTIC DEVICE AND ULTRASOUND IMAGE DISPLAY METHOD

(71) Applicant: Hitachi, Ltd., Tokyo (JP)

(72) Inventors: Taro EGUCHI, Tokyo (JP); Takaya UNO, Tokyo (JP)

(73) Assignee: Hitachi, Ltd., Tokyo (JP)

(21) Appl. No.: 15/557,879

(22) PCT Filed: Oct. 16, 2015
(86) PCT No.: PCT/JP2015/079316

§ 371 Date: Sep. 13, 2017

(30) Foreign Application Priority Data
Mar. 18, 2015 (JP) 2015-055273

(51) Int. Cl. G06F 19/00; G06F 3/0488; G06F 3/041

(52) U.S. Cl. CPC G06F 19/3406; A61B 8/14; G06F 3/0412; G06F 3/04886

(57) ABSTRACT

In the present invention, an ultrasound image is displayed within the display area of a touch panel monitor. Upon receipt of a display instruction, a virtual keyboard is displayed in the display area. The virtual keyboard is displayed on a screen in front of the ultrasound image, there being partial overlap between the virtual keyboard and the ultrasound image. The touch panel monitor is provided with a touch sensor, and an operation involving touching the virtual keyboard is sensed as a keystroke on the virtual keyboard. Since the virtual keyboard is an image that can be transparently displayed, it is possible to observe the ultrasound image through the virtual keyboard as a background image.

### TECHNICAL FIELD

The present disclosure relates to an ultrasound diagnostic device, in particular, an ultrasound diagnostic device with use of a virtual keyboard.

### BACKGROUND

An ultrasound diagnostic device forms an ultrasound image based on received signals by transmitting and receiving ultrasound waves to and from a living body. In ultrasound diagnostic devices, a patient ID, comments, and other data may be entered by using a hardware keyboard or a virtual keyboard (software keyboard) displayed on a display.

In an ultrasound system disclosed in Patent Literature 1, a keyboard image is displayed on a part of a display screen.

### CITATION LIST

### Patent Literature

Patent Literature 1: JP 2010-17558 A

### SUMMARY

### Technical Problem

Due to a limited size of a display screen, a virtual keyboard may overlap an ultrasound image depending on the size of the screen, causing an issue that because the ultrasound image is covered by the virtual keyboard, observation of the ultrasound image is disabled. In order to address this issue, the ultrasound image and the virtual keyboard may be reduced in size to be displayed to avoid the overlap of the ultrasound image and the virtual keyboard. However, this may cause another issue that the observation of the ultrasound image and an input operation using the keyboard become inconvenient.

An object of the present invention is to allow an input by using a virtual keyboard and an observation of an ultrasound image to be performed properly in an ultrasound diagnostic device.

### Solution to Problem

An ultrasound diagnostic device according to the present disclosure includes a display that displays an ultrasound image formed based on a reception signal obtained by transmitting and receiving ultrasound waves; a display controller that performs control to display a virtual keyboard which can be transparently displayed on a display screen on which the ultrasound image is displayed; and a sensor that senses an input to the virtual keyboard on the display screen.

Because the virtual keyboard has transparency, when the virtual keyboard is displayed over an ultrasound image, the ultrasound image can be observed as a background image through the virtual keyboard. In this way, because it is unnecessary to reduce the size of the ultrasound image and the virtual keyboard to avoid an overlapping display of the virtual keyboard and the ultrasound image in a limited display area, it is possible to largely display each display element. Thus, an input operation can be properly performed through the virtual keyboard while properly observing the ultrasound image. It is also possible to avoid selecting and displaying each display element in the display area.

It is preferable that a degree of transparency of the virtual keyboard is variable. In this way, the degree of transparency of the virtual keyboard can be changed when necessary, to observe the ultrasound image or input to the virtual keyboard.

It is further preferable that the display controller shifts the virtual keyboard in the up direction or the down direction on the display screen in accordance with a shift instruction. For example, the virtual keyboard may be displayed by avoiding a region of interest on the ultrasound image.

It is further preferable that the display controller displays a button image on the display to shift the virtual keyboard in the up direction or the down direction. In this way, the display position of the virtual keyboard can be changed by a simple operation.

It is further preferable that when the degree of transparency of the virtual keyboard satisfies a predetermined condition, the sensor senses an input within a display area of the virtual keyboard as an input to a background image behind the virtual keyboard. In this way, an error input to the virtual keyboard can be prevented and the input is sensed as a valid input to the background image.

It is further preferable that the ultrasound diagnostic device further includes a distance sensor that senses a distance between the display and a user. The display controller changes the degree of transparency of the virtual keyboard in accordance with the sensed distance. In this way, the visibility of the virtual keyboard and the ultrasound image is changed in accordance with a user (observer) state.

It is further preferable that the display controller increases the degree of transparency of the virtual keyboard when the distance between the display and the user is larger, compared to the degree of transparency when the distance between the display and the user is smaller. In this way, when the user such as an observer approaches the display, the degree of transparency decreases, enhancing the visibility of the virtual keyboard. Conversely, when the user is farther away from the display, the degree of transparency increases, lowering the visibility of the virtual keyboard but enhancing the visibility of the ultrasound image.

It is further preferable that the display controller changes the display position of the virtual keyboard in accordance with the position of a point of interest or region of interest set for the ultrasound image.

A method for displaying an ultrasound image according to the present disclosure includes displaying an ultrasound image formed based on a reception signal obtained by transmitting and receiving ultrasound waves and performing control to display a virtual keyboard which can be transparently displayed on a display screen on which the ultrasound image is displayed; and sensing an input operation to the virtual keyboard on the display screen.

### Advantageous Effects of Invention

According to the present disclosure, an input by using a virtual keyboard and an observation of an ultrasound image can be performed properly in an ultrasound diagnostic device.

### BRIEF DESCRIPTION OF DRAWINGS

FIG. 1 is a conceptual diagram showing a preferable embodiment of an ultrasound diagnostic system according to the present disclosure;

FIG. 2 is a perspective diagram of an ultrasound diagnostic system in a separated state;

FIG. 3 is a perspective diagram of an ultrasound diagnostic system in a docked state;

FIG. 4 is a block diagram of a front-end device;

FIG. 5 is a block diagram of a back-end device;

FIG. 6 is a table showing communication systems in a docked state and a separated state;

FIG. 7 is a block diagram showing a configuration for display control of a touch panel monitor;

FIG. 8 is a diagram showing a layered structure of images;

FIG. 9 is a diagram showing a first display example of a virtual keyboard;

FIG. 10 is a diagram showing a second display example of a virtual keyboard;

FIG. 11 is a diagram showing a second display example of a virtual keyboard;

FIG. 12 is a diagram showing a second display example of a virtual keyboard;

FIG. 13 is a diagram showing a second display example of a virtual keyboard;

FIG. 14A is a diagram showing a third display example of a virtual keyboard;

FIG. 14B is a diagram showing a third display example of a virtual keyboard;

FIG. 14C is a diagram showing a third display example of a virtual keyboard;

FIG. 15A is a diagram showing a fourth display example of a virtual keyboard;

FIG. 15B is a diagram showing a fourth display example of a virtual keyboard; and

FIG. 16 is a diagram showing another configuration for display control of a touch panel monitor.

### DESCRIPTION OF EMBODIMENTS

Preferred embodiments of the present disclosure are described below by referring to the drawings.

(1) Ultrasound Diagnostic System

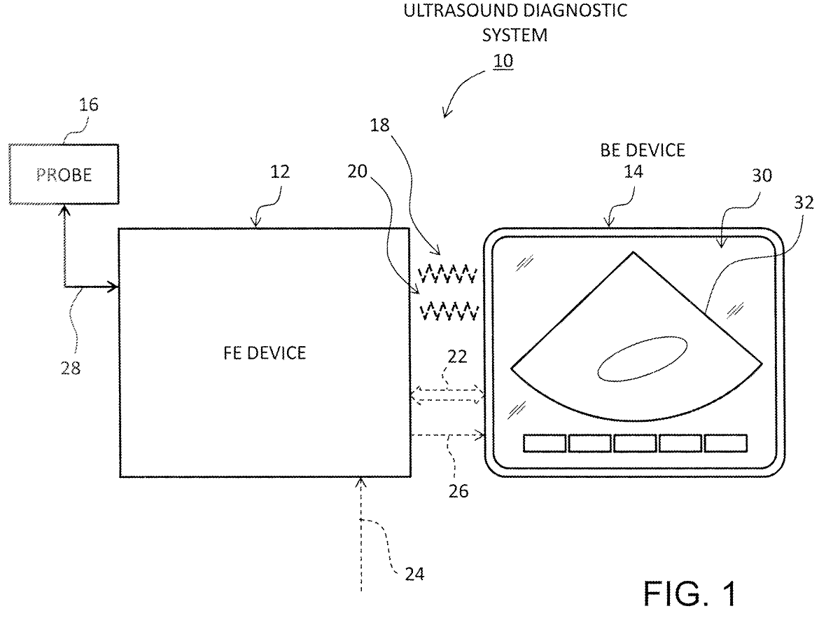

FIG. 1 shows a schematic diagram of an ultrasound diagnostic system according to the present disclosure. An ultrasound diagnostic system 10 is a medical device used at medical facilities such as a hospital, to perform an ultrasound diagnosis to a subject (living body). The ultrasound diagnostic system 10 is configured with, roughly grouped, a front-end (FE) device 12, a back-end (BE) device 14, and a probe 16. The FE device 12 is located near from the living body, whereas the BE device 14 is located far from the living body. The FE device 12 and the BE device 14 are separately-provided portable devices. The FE device 12 and the BE device 14 are operable in a separated state in which these devices are separated, and also operable in a docked state in which these devices are in the combined docked state. FIG. 1 shows a separated state.

The probe 16 is a wave transceiver which transmits and receives ultrasound waves with the probe 16 in contact with a surface of a living body. The probe 16 includes 1D array transducer elements which are arranged in a linear or arch shaped line. The array transducer elements form ultrasound beams which are repeatedly electronically scanned. A beam scan plane is formed in a living body in each electronic scan. As an electronic scanning method, an electronic linear scanning method, an electronic sector scanning method, and other methods are known. In place of the 1D array transducer elements, 2D array transducer elements, which can form a three-dimensional echo data acquisition space, may be used. In the configuration shown in FIG. 1, the probe 16 is connected to the 1th device 12 via a cable 28. The probe 16 may be connected to the FE device 12 through wireless communications. In that case, a wireless probe is used. By connecting two or more probes to the FE device 12, the probe 16 which is to be actually used may be selected from the connected probes. The probe 16 which is to be inserted into a body cavity may be connected to the FE device 12.

In the separated state shown in FIG. 1, the FE device 12 and the BE device 14 are electrically connected to each other through a wireless communication system. In this embodiment, these devices are connected to each other through a first wireless communication system and a second wireless communication system. FIG. 1 clearly shows a wireless communication path 18 in the first wireless communication system and another wireless communication path 20 in the second wireless communication system. The first wireless communication system is faster than the second wireless communication system. In the present embodiment, the first wireless communication system is used to transmit ultrasound reception data from the FE device 12 to the BE device 14. In other words, the first wireless communication system is used for data transmission. In the present embodiment, the second wireless communication system is slower and simpler than the first wireless communication system. The second wireless communication system is used to transmit control signals from the BE device 14 to the FE device 12. In other words, the second wireless communication system is used for control.

In the docked state in which the FE device 12 and the BE device 14 are physically connected to each other, the FE device 12 and the BE device 14 are electrically connected through a wired communication system. The wired communication system is significantly faster than the above two wireless communication systems. FIG. 1 shows a wired communication path 22 between the two devices. A power supply path 26 is used to supply DC electrical power from the FE device 12 to the BE device 14. The supplied electrical power is used for operating the BE device 14 and for charging a battery in the BE device 14.

Reference numeral 24 represents a DC power supply line from an AC adaptor (AC/DC converter). The AC adaptor is connected to the FE device 12, if necessary. Because the FE device 12 has a built-in battery, the FE device 12 can operate by using the battery as a power source. The FE device 12 has a box-like shape as described below. The configuration and operations of the FE device 12 are described in detail below.

In contrast, the BE device 14 has a tablet-like or plate-like shape in the present embodiment. The BE device 14 basically has a configuration similar to a general tablet computer, except that the BE device 14 is mounted with various dedicated software for ultrasound diagnosis. Such software includes an operation control program, an image process program, and other programs. The BE device 14 includes a display panel 30 with a touch sensor. The display panel 30 functions as a user interface, serving both as an input device and a display. In FIG. 1, a B-mode tomographic image 32 is displayed on the display panel 30 as an ultrasound image. A user performs various inputs by using icons displayed on the display panel 30. It is possible to perform a slide operation and a zoom-in operation on the display panel 30.

The ultrasound diagnostic system 10 can be operated in a usage state selected from a separated state and a docked state, in accordance with a diagnostic application and the preference of a diagnostician. Therefore, a highly-usable ultrasound diagnostic system can be provided.

In order to avoid the ultrasound diagnostic system 10 becoming unstable or improper when the state is changed, the ultrasound diagnostic system 10 is controlled to be forced into a freeze state when the state is changed. Specifically, in a transaction process from the separated state to the docked state, an immediately-before-docked state is determined respectively in the FE device 12 and the BE device 14 based on the wave strength and the reception state, which are indicators of the distance between the two devices. Based on this determination, the FE device 12 and the BE device 14 are respectively controlled to switch the operation state to the freeze state. The freeze state of the FE device 12 and the BE device 14 is released after the docked state is achieved and the diagnostician has performed a freeze release operation. In a transition state from the docked state to the separated state, the separated state is detected respectively in the FE device 12 and the BE device 14 by detecting a pulled-out wire or by another method to place the devices in the freeze state. Then, the freeze state of the FE devices FE device 12 and 14 is released after the freeze release operation.

The BE device 14 may be separately connected to a hospital LAN by a wireless communication system or a wired communication system. Such a communication path is omitted in the drawing. The BE device 14 (or the FE device 12) may be separately connected to another dedicated device (such as a remote controller) used for ultrasound diagnosis by a wireless communication system or a wired communication system.

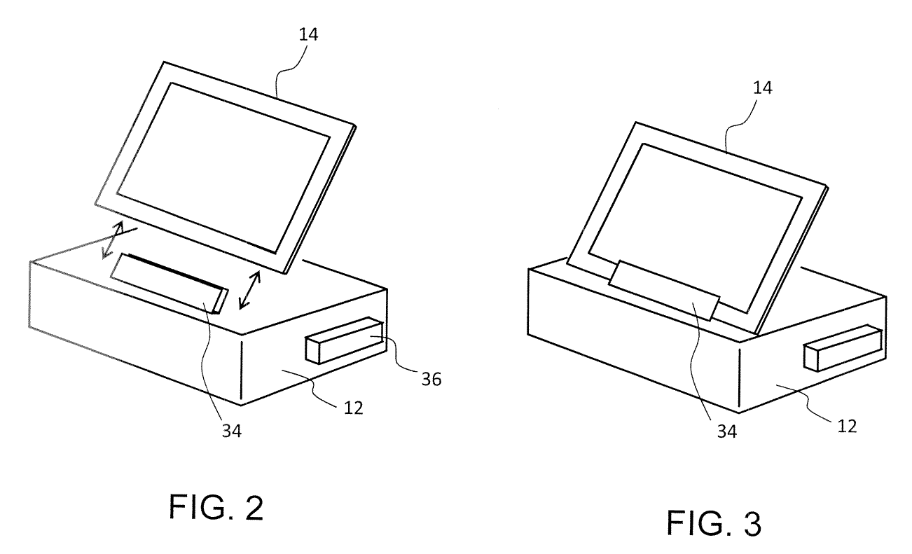

FIG. 2 shows a separated state. The FE device 12 may be placed, for example, on a desk. The FE device 12 includes a holder 34 which has an insertion port (slot). The holder 34 has a hinge mechanism such that the holder 34 is pivotable about a horizontal axis. On end of a probe cable is connected to a predetermined side of the FE device 12. A chamber for accommodating a probe or other elements may be formed inside the FE device 12. Such a structure is convenient when carrying the ultrasound diagnostic system, and the probe can be protected. In FIG. 2, the BE device 14 is separated from the FE device 12. The BE device 14 may be further separated from the FE device 12 within a wirelessly communicable range.

FIG. 3 shows a docked state. A bottom edge portion of the BE device 14 is inserted into the insertion port of the holder 34. In this inserted state, the FE device 12 and BE device 14 are in wired communication with each other. In other words, the devices are both connected through a wired LAN and a wired power supply line. In the docked state, the angle of the BE device 14 may be arbitrary changed to adjust the orientation. The BE device 14 may be tilted completely to the back surface side (to the upper surface side of the FE device 12).

(2) Front-End Device

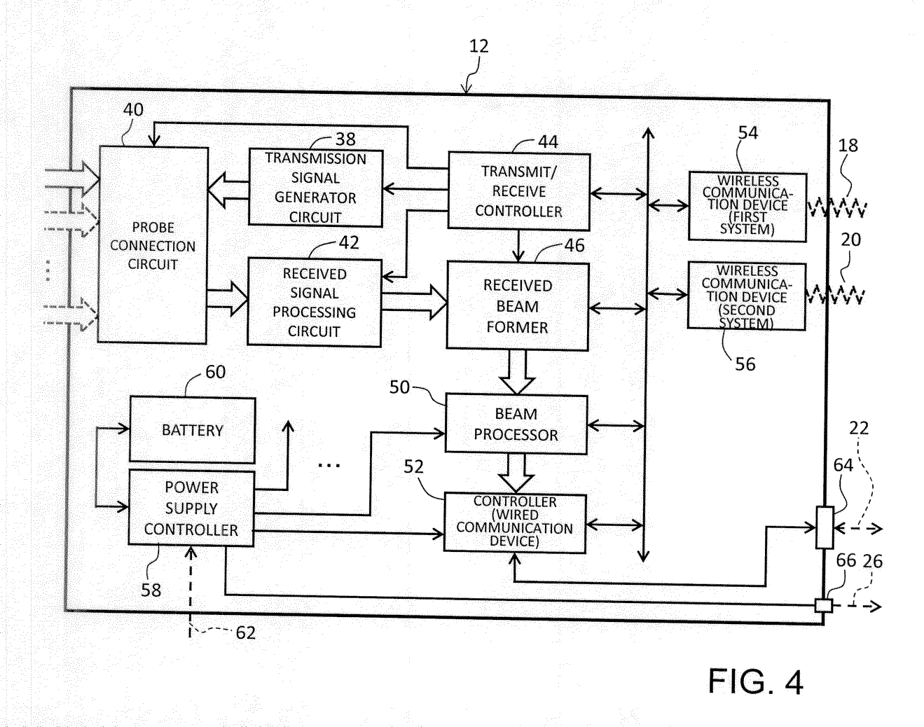

FIG. 4 is a block diagram of the FE device 12. The respective blocks in the drawing are configured with hardware such as processors and electronic circuits. A transmission signal generator circuit 38 supplies two or more transmission signals in parallel to two or more transducer elements in the probe. In response to the supplied signals, transmission beams are generated by the probe. When the two or more transducer elements receive reflected waves from inside a living body, received signals are output from those transducer elements such that the received signals are input to a received signal processing circuit 42 via a probe connection circuit 40. The received signal processing circuit 42 includes preamplifiers, amplifiers, A/D converters, and other elements. Digital received signals which have been output from the received signal processing circuit 42 are sent to a received beam former 46. The received beam former 46 applies a phase addition process to the digital received signals and outputs beam data as signals after the phase addition. The beam data are formed with two or more echo data items which correspond to the received beam and are arranged in the depth direction. Received frame data are formed with two or more beam data items which have been obtained in a single electronic scan.

A transmit/receive controller 44 controls generation of transmission signals and processing of received signals based on transmit/receive control data which have been sent from the BE device 14. A beam processor 50 is a circuit which applies various data processing such as a detection process, a log conversion process, a correlation process etc., to respective beam data which are time-sequentially input. A controller 52 controls overall operations of the FE device 12. Besides such controls, the controller 52 also performs controls for sending, to the BE device 14, beam data which have been sequentially sent from the beam processor 50 either via cable or wirelessly. In the present embodiment, the controller 52 also functions as a wired communication device. A wireless communication device 54 is a module which communicates in a first wireless communication system. A wireless communication device 56 is another module which communicates in a second wireless communication system. Reference numeral 18 represents a wireless communication path in the first wireless communication system. Reference numeral 20 represents a wireless communication path in the second wireless communication system. Although the paths are both bidrectional communication paths, in the present embodiment, vast received data are transmitted from the FE device 12 to the BE device 14 by using the wireless communication path 18, whereas control signals are transmitted from the BE device 14 to the FE device 12 by using the wireless communication path 20. Reference numeral 64 represents a wired communication terminal to which the wired communication path 22 is connected. Reference numeral 66 represents a power supply terminal to which the power supply line 26 is connected. The power supply line 26 supplies DC power from the FE device 12 to the BE device 14 as described above.

A battery 60 may be, for example, a lithium ion type battery. The charge and discharge of the battery 60 are controlled by a power supply controller 58. When the battery is in operation, electrical power from the battery 60 is supplied to each circuit in the FE device 12 via the power supply controller 58. Reference numeral 62 represents a power supply line when an AC adaptor is connected. When the AC adaptor is connected, external electrical power is supplied to each circuit in the FE device 12 through the operation of the power supply controller 58. During this process, when the charge amount of the battery 60 is below 100%, the battery 60 is charged by the external electrical power.

During an ultrasound diagnosis operation (during transmission and reception), the FE device 12 repeatedly supplies transmission signals to a probe and processes received signals which are subsequently received. The time-sequence beam data which are obtained by these repeated processes are sequentially transmitted to the BE device 14 through wireless communication in the separated state or through wired communication in the docked state. During this process, respective beam data are converted into packets such that respective beam data are transmitted in a so-called packet transmission system.

Besides the B mode, various modes are known as operation modes, including a CMF mode, an M mode, and D modes (a PW mode and a CW mode). Transmission/reception processes may be performed for harmonic imaging and elasticity information imaging. In FIG. 4, a circuit such as a living body signal input circuit is omitted.

(3) Back-End Device

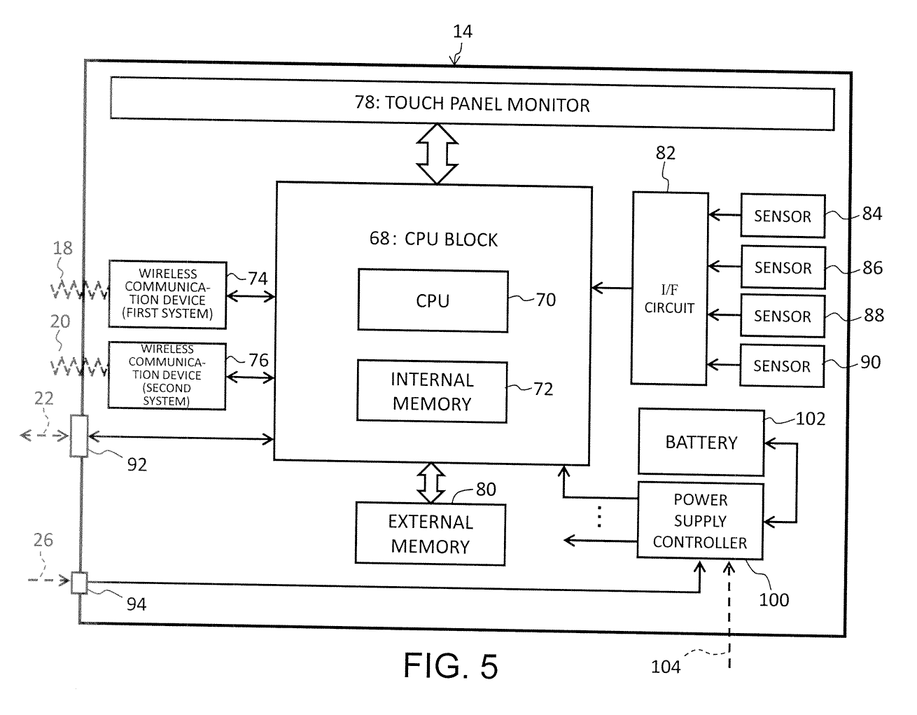

FIG. 5 is a block diagram of the BE device 14. In the drawing, each block indicates hardware such as a processor, a circuit, and a memory. A CPU block 68 includes a CPU 70 and an internal memory 72. The internal memory 72 serves as a working memory or a cache memory. An external memory 80, which is connected to the CPU block 68, stores various types of programs such as an OS, control programs and processing programs. The processing programs include a scan convert processing program. The external memory 80 also serves as a cine memory having a ring buffer configuration. The cine memory may be formed in the internal memory 72.

The CPU block 68 generates display frame data by a scan convert process based on two or more beam data items. The display frame data form an ultrasound image (for example, a tomographic image). The display frame data are sequentially processed to produce video. The CPU block 68 applies, to the beam data or images, various processes to display ultrasound images. Besides these processes, the CPU block 68 controls operations of the BE device 14 and the entire ultrasound diagnostic system.

A touch panel monitor (display panel) 78 serves as an input device and a display device. Specifically, the touch panel monitor 78 includes a liquid crystal display and a touch sensor to serve as a user interface. The touch panel monitor 78 displays images, including ultrasound images. A virtual keyboard (software keyboard) and various types of buttons (icons) for operations are also displayed.

A wireless communication device 74 is a module for wireless communications in the first wireless communication system. The wireless communication path for such communications is shown by reference numeral 18. Another wireless communication device 76 is a module for wireless communications in the second wireless communication system. The wireless communication path for such communications is shown by reference numeral 20. The CPU block 68 is also provided with a function for wired communications so as to communicate in accordance with a wired communication system. In a docked state, a wired communication line is connected to a wired communication terminal 92. A power supply line 26 is connected to a power supply terminal 94.

The CPU block 68 includes two or more sensors 84 to 90 connected thereto via an interface (I/F) circuit 82. The sensors may include an illumination sensor, a proximity sensor, a temperature sensor, and a distance sensor. A module such as a GPS may be connected. The I/F circuit 82 serves as a sensor controller.

A battery 102 is a lithium ceramic type battery. The charge and discharge of the battery are controlled by a power supply controller 100. The power supply controller 100 supplies electrical power from the battery 102 to the respective circuits in the BE device 14 when the battery is in operation. When the battery is not in operation, the power supply controller 100 supplies the electrical power from the FE device 12 or an AC adaptor to the respective circuits in the BE device 14. Reference numeral 104 represents a power supply line via the AC adaptor.

The BE device 14 generates ultrasound images by sequentially processing the beam data fed from the FE device 12 while controlling the FE device 12 so as to display the generated ultrasound images on the touch panel monitor 78. During this process, a graphic image used for operation is also displayed together with the ultrasound images. In a usual real-time operation, the BE device 14 and the FE device 12 are electrically connected to each other by a cable or wirelessly. The devices are synchronized while the ultrasound diagnostic operation is continuously performed. In a freeze state, operations of a transmission signal generation circuit and a reception signal generation circuit are stopped, and operations of a boost circuit in the power supply controller 100 are also stopped. In the BE device 14, the display turns to a still-image display at the time of freezing to maintain the displayed image. The BE device 14 may be configured to be connectable with an external display.

(4) Communication Systems

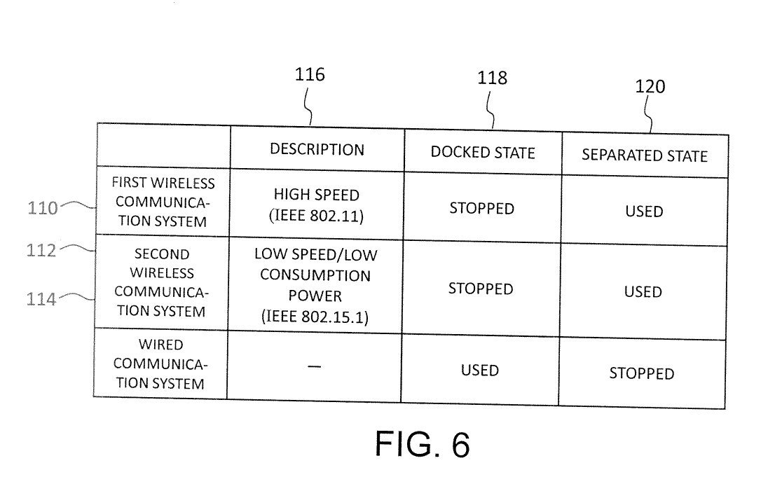

FIG. 6 shows, in an organized manner, communication systems used in a docked state 118 and a separated state 120. Reference numeral 110 represents a first wireless communication system, whereas reference numeral 112 represents a second wireless communication system. Reference numeral 114 represents a wired communication system. Reference numeral 116 represents descriptions of the wireless communication systems. In the docked state 118, wired communication is selected such that the first wireless communication device and the second wireless communication device in the FE device 12 and the BE device 14 are placed in an operation stopped state. This can achieve energy saving. Conversely, in the separated state 120, wireless communications are selected such that the first wireless communication device and the second wireless communication device are operated in the FE device 12 and the BE device 14. During this process, the wired communication system is placed in an operation stopped state. It should be noted that the first wireless communication system 110 is faster than the second wireless communication system 112. Conversely, the second wireless communication system 112 is slower than the first wireless communication system 110, but the second wireless communication system 112 is simpler, less costly, and consumes less power. As the wired communication system, a TCP/IP protocol on Ethernet (registered trademark) is available. As the first wired communication system and the second wired communication system, IEEE 802.11 and IEEE 802.15.1 are respectively available. These communication systems are listed merely as examples. Other communication systems may be used. In either case, it is desirable to use a secured communication system.

In the present embodiment, the wireless communication device in the second wireless communication system 112 is provided with a function to automatically change the transmission power in accordance with a reception strength (in other words, distance). Specifically, when the FE device 12 is placed in the vicinity of the BE device 14, control is automatically performed to reduce the transmission power of both of the devices. Therefore, it is possible to determine that the devices are placed in the vicinity of each other based on the set transmission power. Alternatively, it is also possible to determine that the two devices are placed in the vicinity of each other based on other factors such as the reception strength and reception error rate. Further, a proximity sensor may be used. In the above configuration, the BE device 14 itself serves as the ultrasound diagnostic device. A system in which the FE device 12 and the BE device 14 are combined also serves as the ultrasound diagnostic device.

(5) Virtual Keyboard

The BE device 14 is provided with a function to display a virtual keyboard. This function is described below. In the present embodiment, the virtual keyboard (software keyboard) is displayed on the touch panel monitor 78, when necessary. The virtual keyboard is a keyboard which accepts an input from a user on the touch panel monitor 78.

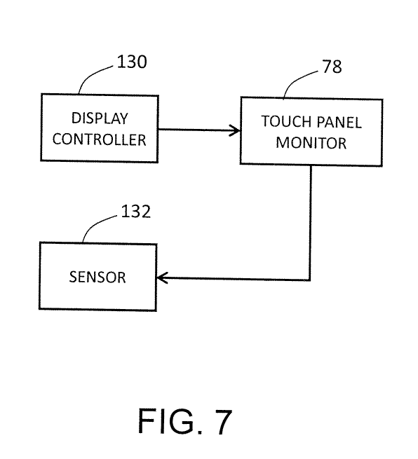

FIG. 7 shows a configuration of display control of a touch panel monitor. A display controller 130 displays ultrasound images, various buttons (icons) for operations, a virtual trackpad, a virtual keyboard, and others on the touch panel monitor 78. For example, the display controller 130 displays the virtual keyboard in a display area where an ultrasound image is displayed on the touch panel monitor 78. The data of this virtual keyboard are stored in advance in an internal memory 72 in a CPU block 68, or in the external memory 80.

The virtual keyboard according to the present embodiment is an image which can be transparently displayed with a variable degree of transparency. The degree of transparency is a variable value within a range from 0% to 100%. This degree of transparency may be set by a user or automatically. When the degree of transparency is set to “0%”, the display controller 130 displays the virtual keyboard in a completely-opaque state on the touch panel monitor 78. In this case, it is impossible to view a background image through the virtual keyboard. Conversely, when the degree of transparency is set to “100%”, the virtual keyboard becomes completely transparent. In this case, it is impossible to view the virtual keyboard. A degree of transparency closer to “0%” makes the degree of transparency of the virtual keyboard closer to opaque, whereas a degree of transparency closer to “100%” makes the degree of transparency of the virtual keyboard closer to complete transparency. How the background image appears varies in accordance with the degree of transparency.

A sensor 132 is a touch sensor for sensing a touch operation (input) on the touch panel monitor 78. As a sensing system of the touch panel monitor 78, a well-known system can be applied. As a typical system, a capacitive system or a resistive film system may be used. The sensor 132 senses operations such as a drag operation in which a touch position is moved on the touch panel monitor 78 and a release operation in which the touch is detached from the touch panel monitor 78. For example, the sensor 132 senses operations such as a touch operation and a drag operation on a virtual trackpad. The sensor 132 further senses touch operations on the various buttons. When the virtual keyboard is displayed on the touch panel monitor 78, the sensor 132 senses touch operations on the virtual keyboard. The sensor 132 senses touch operations to respective elements on the virtual keyboard to accept input of text and instructions.

The display controller 130 and the sensor 132 may be provided as functions of, for example, the CPU block 68 in the BE device 14.

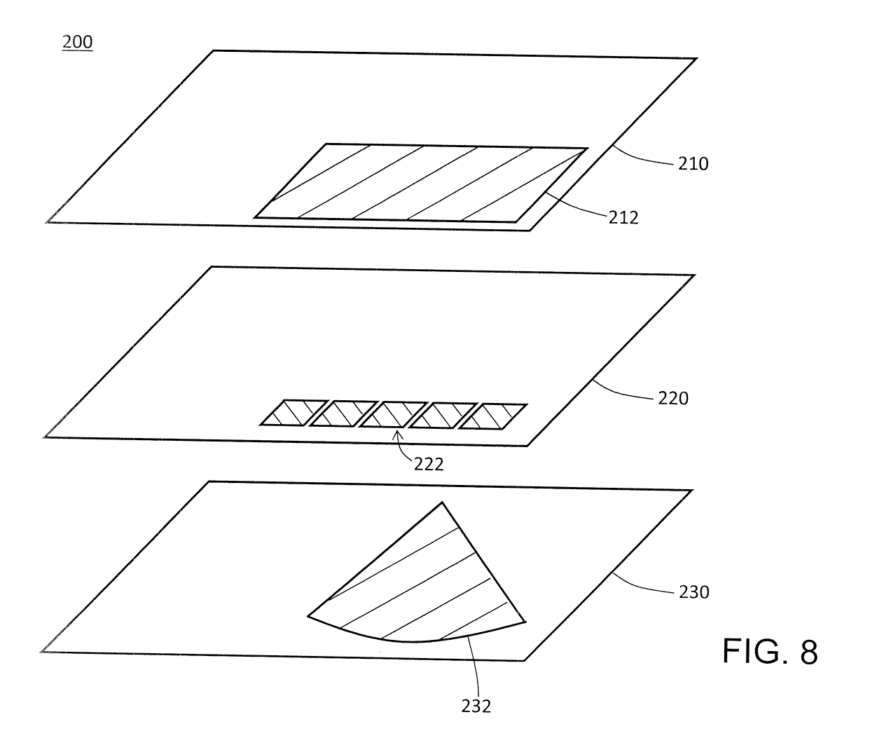

FIG. 8 shows an example of a layer structure of images displayed on the touch panel monitor 78. A layer structure 200 includes two or more overlapping layers (hierarchy). A layer 210 includes a virtual keyboard 212. A layer 220 includes a button group 222 (icon group) for operation. A layer 230 includes an ultrasound image 232 (image such as a B-mode tomographic image). As an example, the layer 210 is the front layer, the layer 220 is the middle layer, and the layer 230 is the back layer. This arrangement is, of-course, only an example. The layers may be arranged in any other order.

For example, when a display instruction for the ultrasound image 232 is provided while no display instruction for the virtual keyboard 212 and the button group 222 is provided, the display controller 130 displays the layer 230 on the touch panel monitor 78. In this way, the ultrasound image 232 is displayed on the touch panel monitor 78. When a display instruction for the virtual keyboard 212 is provided in this state, the display controller 130 displays the layer 210 over the layer 230 on the touch panel monitor 78. The ultrasound image 232 becomes a background image of the virtual keyboard 212. When the display position of the ultrasound image 232 and the display position of the virtual keyboard 212 are overlapped, the images are displayed such that the virtual keyboard 212 overlaps the ultrasound image 232 at the overlapping portion. The display controller 130 sets the degree of transparency of the layer 210 as designated. In this way, the degree of transparency of the virtual keyboard 212 is set such that the degree of transparency changes how the ultrasound image 232 appears in the area overlapped with the virtual keyboard 212. When the degree of transparency is set to “0%”, it is impossible to view the ultrasound image 232 in the overlapping portion. When the degree of transparency is set to “100%”, the virtual keyboard 212 becomes completely transparent such that it becomes possible to view the ultrasound image 232 while the virtual keyboard 212 is invisible. When the degree of transparency is set to a value between 0 and 100%, how the overlapping portion appears changes in accordance with the set value.

When display instructions for the ultrasound image 232 and the button group 222 are provided while no display instruction for the virtual keyboard 212 is provided, the display controller 130 displays the layer 220 over the layer 230 on the touch panel monitor 78. In this way, the ultrasound image 232 and the button group 222 are displayed. When the display position of the ultrasound image 232 and the display position of the button group 222 are overlapped, the images are displayed such that the button group 222 overlaps the ultrasound image 232 at the overlapping portion.

When display instructions for the ultrasound image 232, the virtual keyboard 212, and the button group 222 are provided, the display controller 130 displays the layer 220 over the layer 230, and the layer 210 over the layer 220 on the touch panel monitor 78.

It should be noted that the layer structure 200 shown in FIG. 8 is merely an example. One or more layers other than the layers 210 to 230 may be included in the layer structure 200.

The virtual keyboard is described in detail below.

First Display Example

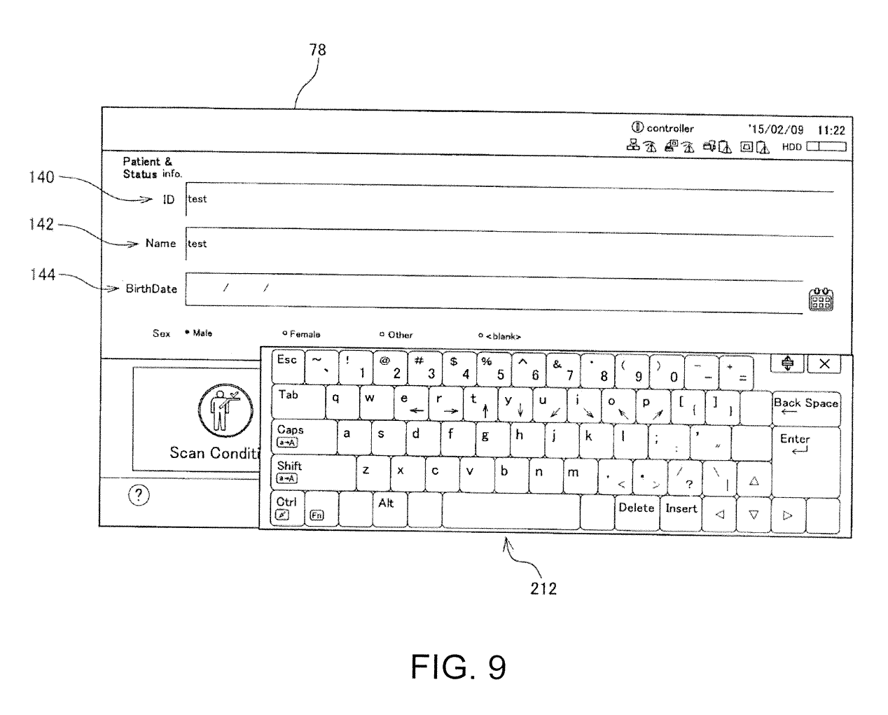

Referring to FIG. 9, the first display example of a virtual keyboard is described. FIG. 9 shows a display area of the touch panel monitor 78 when inputting a patient ID. When inputting a patient ID, the display controller 130 displays a patient ID input field 140, a patient name input field 142, a patient birthday input field 144, a gender selection field, and the like. When an input field is designated by a user (for example, by a touch operation) in this state, the display controller 130 displays the virtual keyboard 212 on the touch panel monitor 78. In this way, an input of the patient ID or the like using the virtual keyboard 212 becomes possible. For example, the display controller 130 displays the virtual keyboard 212 in an area avoiding the display position of the input fields 140 to 144. In the example shown in FIG. 9, because the input fields 140 to 144 are displayed in the upper portion in the display area, the virtual keyboard 212 is displayed in the lower portion in the display area. When the input fields 140 to 144 are displayed in the lower portion in the display area, the virtual keyboard 212 may be displayed in the upper portion in the display area. Because no image overlaps the virtual keyboard 212 when inputting the patient ID, the display controller 130 sets the degree of transparency of the virtual keyboard 212 to “0%”. In this way, the virtual keyboard 212 is displayed in a completely opaque state. The display controller 130 may, of course, display the virtual keyboard 212 at any position in accordance with an instruction from the user and sets the degree of transparency of the virtual keyboard 212 to a value designated by the user.

Second Display Example

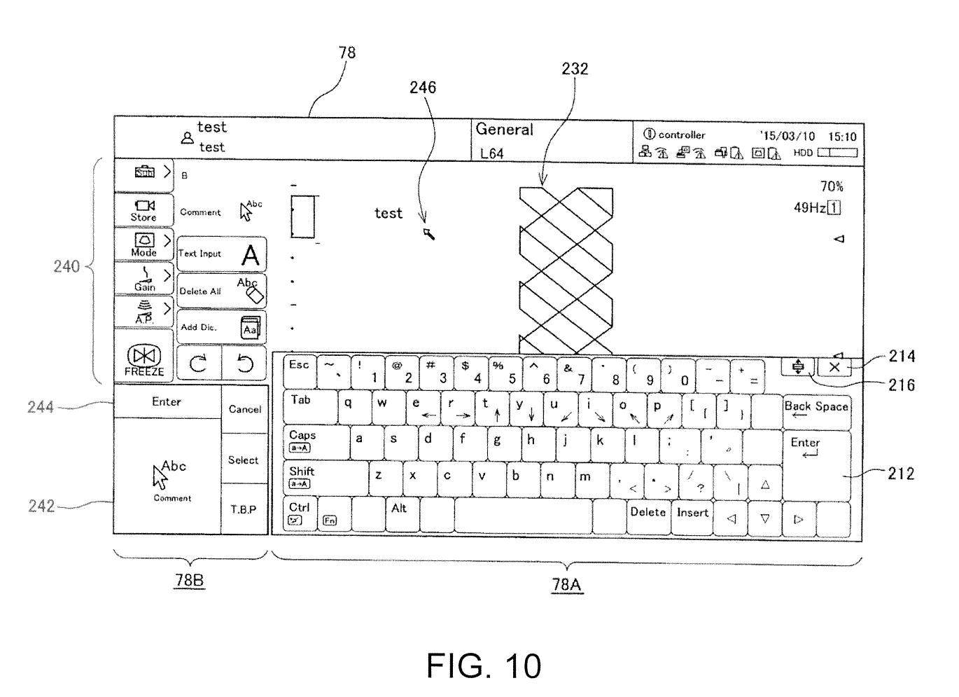

Referring to FIGS. 10 to 13, the second display example of the virtual keyboard is described. FIG. 10 shows a display area of the touch panel monitor 78 during an ultrasound diagnosis. During the ultrasound diagnosis, the display area of the touch panel monitor 78 includes display areas 78A and 78B.

The display area 78A is an examination screen area, in which an ultrasound image 232 (for example, a B-mode tomographic image) is displayed. When a display instruction for the virtual keyboard 212 is provided, the virtual keyboard 212 is displayed in the display area 78A. The virtual keyboard 212 is, for example, a so-called full keyboard provided with function keys and a numeric keypad. Of course, a keyboard without the function keys and numeric keypad may also be used for the virtual keyboard 212. Further, the virtual keyboard 212 may include any keys dedicated for the ultrasound diagnosis. For example, up/down or diagonal arrow keys may be included in the virtual keyboard 212.

The display area 78B is an operation area (command area), in which a button group 240 (icon group) for inputting various commands and a virtual trackpad 242 are displayed. The button group 240 includes buttons such as a mode set button to set an ultrasound diagnosis mode, a freeze button to freeze the ultrasound image, a store button to store the ultrasound image, a gain adjustment button, and a comment input button to input comments. When a user performs a touch operation to any of the buttons, the touch operation is sensed by the sensor 132 (touch sensor) and a process corresponding to the button is performed. The virtual trackpad 242 enables, on a screen, operations similar to those of a trackpad (touchpad). By performing a drag operation on the virtual trackpad 242, a pointer 246 displayed in the display area 78A can be moved in the drag direction for the distance corresponding to the drag amount. Of course, the pointer 246 may be directly moved by performing the drag operation directly on the pointer 246. A button group 244 is displayed around the virtual trackpad 242. The button group 244 includes buttons corresponding to an “Enter key”, a “Cancel” key, and a “Select” key.

In the present embodiment, the virtual keyboard 212 is displayed in the display area 78A, not in the display area 78B, to avoid interference with the button group 240 displayed in the display area 78B.

For example, when a user designates (for example, by a touch operation) the comment input field, the display controller 130 displays the virtual keyboard 212 in the display area 78A of the touch panel monitor 78. In this way, comments can be input through the virtual keyboard 212. A hide button 214 is provided for the virtual keyboard 212. When the user performs a touch operation on the hide button 214, the display controller 130 hides the virtual keyboard 212. Alternatively, the display controller 130 may hide the virtual keyboard 212 when a touch operation is performed in an area other than the virtual keyboard 212.

In a default state, the virtual keyboard 212 is displayed, for example, in a lower area in the display area 78A. The virtual keyboard 212 has a vertical length (length in the height direction) of, for example, less than half the vertical length of the display area 78A. The virtual keyboard 212 is provided with an up/down shift button 216 for shifting the virtual keyboard 212 in the up or down direction (in the height direction). When the user performs a touch operation on the up/down shift button 216 with the virtual keyboard 212 being displayed in the lower area, the display controller 130 moves the virtual keyboard 212 in the up direction to display the virtual keyboard 212 in the upper area. Of course, the virtual keyboard 212 may be displayed in the upper area by default.

The virtual keyboard 212 is displayed in front of the ultrasound image 232 such that the virtual keyboard 212 partially overlaps with the ultrasound image 232. The degree of transparency of the virtual keyboard 212 is set in accordance with a user instruction within a range from 0 to 100%. For example, the display controller 130 displays a transparency degree setting bar (not shown) for setting the degree of transparency from 0 to 100% on the touch panel monitor 78. When the user designates the degree of transparency by a touch operation on the transparency degree setting bar, the display controller 130 sets the degree of transparency of the virtual keyboard 212 to the value designated by the user. In the example shown in FIG. 10, because the degree of transparency of the virtual keyboard 212 is set to “0%”, the virtual keyboard 212 is displayed in a completely opaque state. In this state, the user cannot view the overlapping portion of the ultrasound image 232 through the virtual keyboard 212.

At the same time, an input to the virtual keyboard 212 becomes effective. For example, when a user performs a touch operation on the virtual keyboard 212, the touch operation is sensed by the sensor 132 (touch sensor) such that texts and instructions are input.

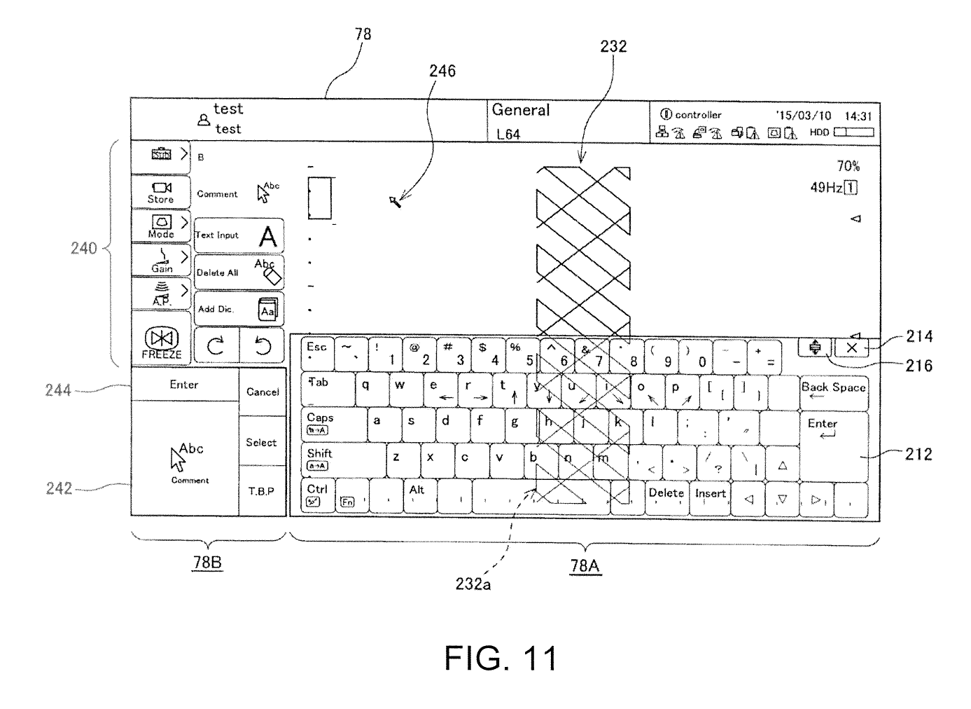

FIG. 11 shows the virtual keyboard 212 which is displayed in a semitransparent state. For example, when the degree of transparency is set to a value between 0 to 100% (excluding 0 and 100%), the virtual keyboard 212 is displayed in a semitransparent state in accordance with the set degree of transparency. In such a state, the user can view the overlapping portion of the ultrasound image 232 through the virtual keyboard 212. The portion indicated by a dashed-line arrow 232a in FIG. 11 is the overlapping portion of the ultrasound image 232 and the virtual keyboard 212. In this portion, because the ultrasound image 232 can be seen through the virtual keyboard 212, the user can observe the portion. At the same time, an input to the virtual keyboard 212 becomes effective. For example, when a user performs a touch operation on the virtual keyboard 212, the touch operation is sensed by the sensor 132 (touch sensor) such that text and instructions are input. In this way, the ultrasound image 232 can be sufficiently observed while a text input is possible through the virtual keyboard 212.

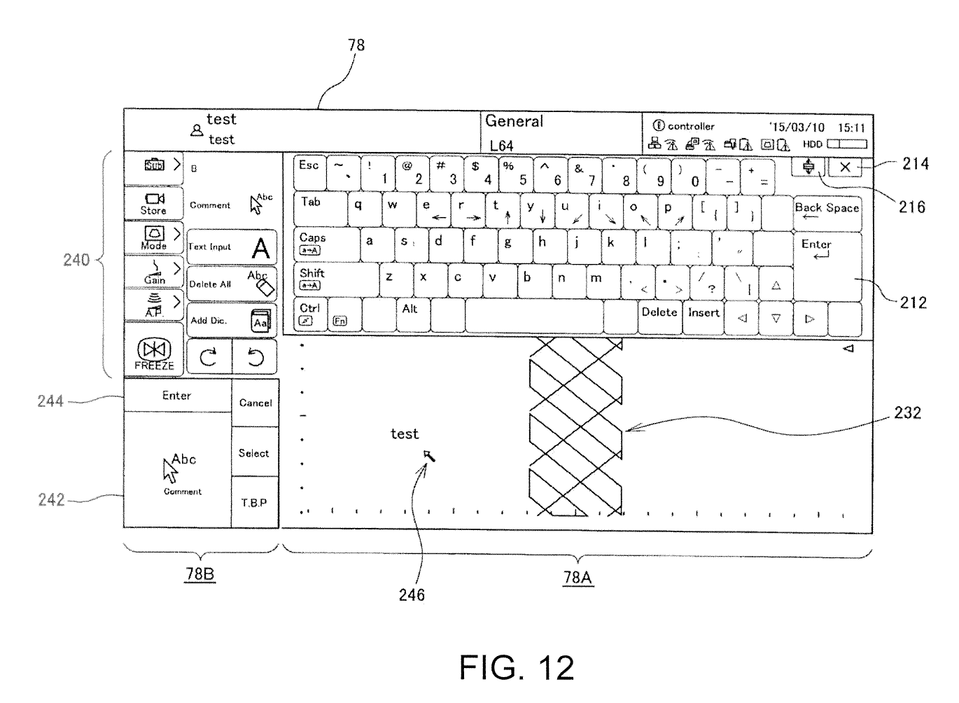

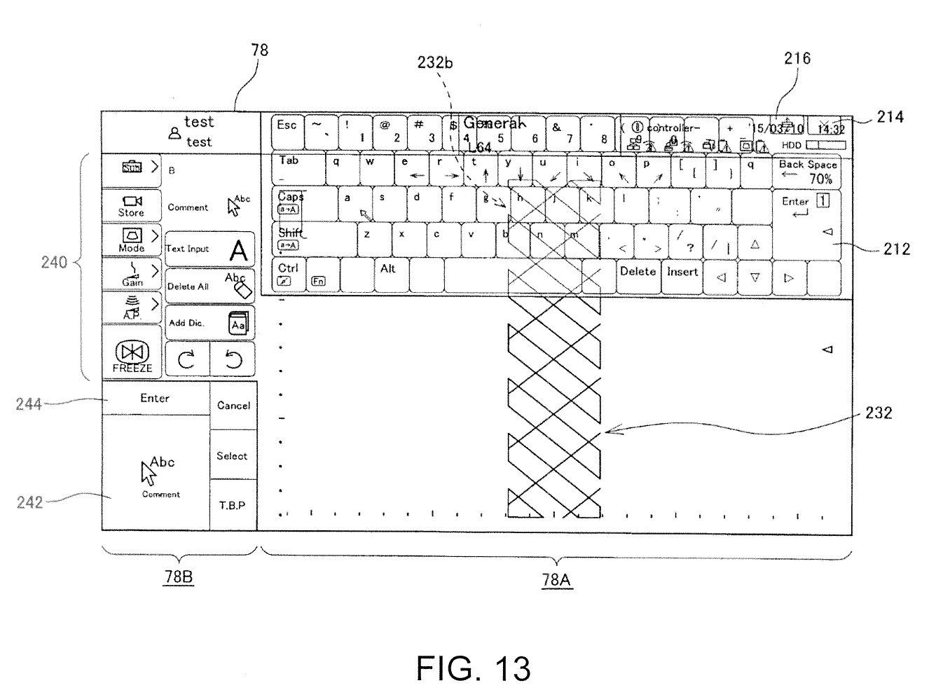

FIGS. 12 and 13 shows the virtual keyboard 212 which is shown in the upper area. In the example shown in FIG. 12, because the degree of transparency of the virtual keyboard 212 is set to “0%”, the virtual keyboard 212 is displayed in a completely opaque state. In this state, the user cannot observe the overlapping portion of the ultrasound image 232 through the virtual keyboard 212.

In the example shown in FIG. 13, the virtual keyboard 212 is displayed in a semitransparent state. In this state, the user can observe the overlapping portion of the ultrasound image 232 through the virtual keyboard 212. The portion indicated by a dashed-line arrow 232b in FIG. 13 is the overlapping portion of the ultrasound image 232 and the virtual keyboard 212. In this portion, because the ultrasound image 232 can be viewed through the virtual keyboard 212, the user can observe the portion. In this way, the ultrasound image 232 can be properly observed while a text input is possible through the virtual keyboard 212.

When the user performs a touch operation on the up/down shift button 216 with the virtual keyboard 212 being displayed in the upper area, the display controller 130 moves the virtual keyboard 212 in the down direction to display the virtual keyboard 212 in the lower area.

For example, the virtual keyboard 212 can be shifted in the upper or lower direction to avoid the overlapping of the virtual keyboard 212 over the region of interest in the ultrasound image 232. In this way, the region of interest can be directly observed, not through the virtual keyboard 212. As described above, in the present embodiment, the vertical length of the virtual keyboard 212 is less than half the vertical length of the display area 78A. Thus, because no overlapping portion exists between the upwardly-shifted virtual keyboard 212 and the downwardly-shifted virtual keyboard 212, the region of interest can be directly observed, not through the virtual keyboard 212, by shifting the virtual keyboard 212 in either direction.

Alternatively, the display controller 130 may display the virtual keyboard 212 in an intermediate area between the upper area and the lower area, or at any position in the vertical direction (height direction).

As described above, in the present embodiment, the display of the virtual keyboard 212 is limited to within the display area 78A, and the movement of the virtual keyboard 212 is limited to the vertical direction. Of course, the virtual keyboard 212 may be configured to be moveable in the horizontal direction or a diagonal direction. For example, when the display area 78A has a sufficiently large size, the virtual keyboard 212 may be configured to be moveable in any direction. It should be noted that a reduced or enlarged display of the virtual keyboard 212 may also be enabled.

As described above, in the present embodiment, the transparently-displayable virtual keyboard 212 is displayed. The transparency of the virtual keyboard 212 enables observation of the ultrasound image 232 through the virtual keyboard 212 when the virtual keyboard 212 is overlappingly displayed over the ultrasound image 232. In this way, in a limited display area 78A, reduction in the display size of the ultrasound image 232 and the virtual keyboard 212 in order to prevent the overlapping display of the ultrasound image 232 and the virtual keyboard 212 can be avoided, enabling display sizes to be made large. Thus, the ultrasound image 232 can be properly observed in a an enlarged state and the input operation can be properly performed by using the virtual keyboard 212 that is displayed enlarged. It becomes unnecessary to select and display the elements to be included in the virtual keyboard 212. This is convenient because the virtual keyboard 212 corresponding to, for example, a full keyboard can be used.

Third Display Example

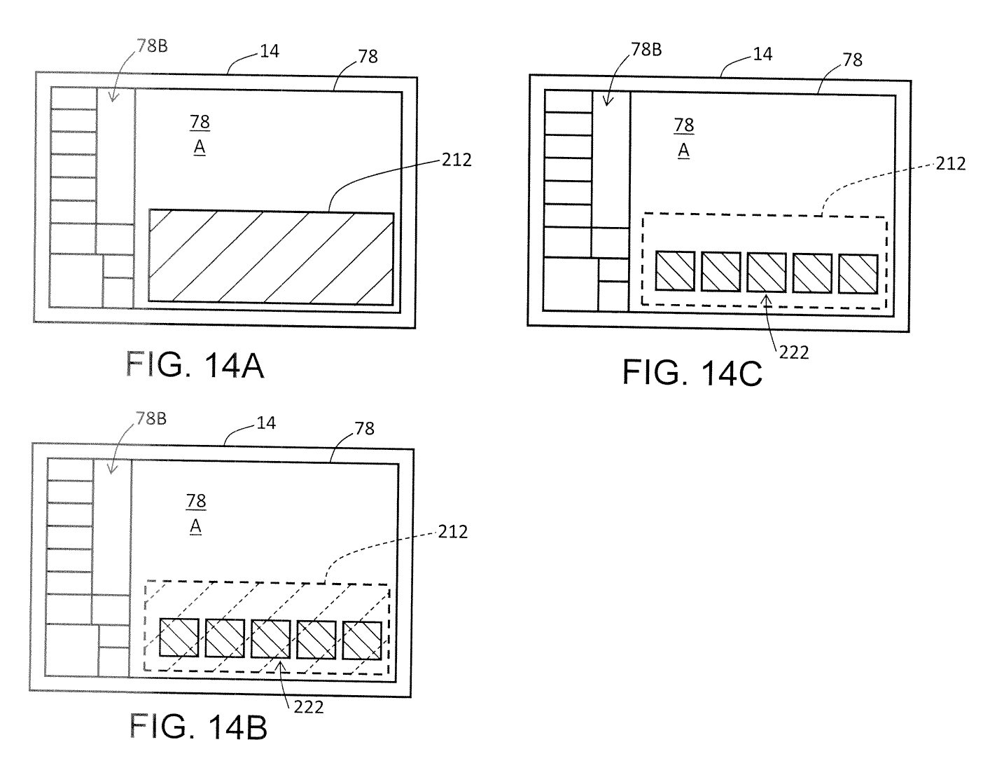

Referring to FIGS. 14A, 14B, and 14C, the third display example of the virtual keyboard is described. In the third display example, in accordance with the degree of transparency, the sensor 132 (touch sensor) may not sense an input on the virtual keyboard, recognizing the input as an invalid input. Instead, the sensor 132 senses an input on the background image of the virtual keyboard as a valid input.

It is assumed here that the layer structure 200 shown in FIG. 8 is applied. Specifically, it is assumed that the layer 220 including the button group 222 is disposed behind the layer 210 including the virtual keyboard 212. Because the display position of the button group 222 and the display position of the virtual keyboard 212 are overlapped, when the layers 210 and 220 are overlappingly displayed, the virtual keyboard 212 is displayed over the button group 222.

FIG. 14A shows the virtual keyboard 212 in “0%” degree of transparency. This virtual keyboard 212 is displayed in the lower area in the display area 78A. Although an ultrasound image is not displayed, for the sake of convenience of description, an ultrasound image is displayed in the display area 78A as shown in FIG. 10. Because the degree of transparency is set to “0%”, the virtual keyboard 212 is displayed in a completely opaque state. Thus, the button group 222 behind the virtual keyboard 212 cannot be viewed. In this state, the sensor 132 (touch sensor) senses a touch operation on the virtual keyboard 212 as a valid key input to the virtual keyboard 212. Thus, an input by using the virtual keyboard 212 is performed.

By increasing the degree of transparency of the virtual keyboard 212, it becomes possible to view the button group 222 on the background through the virtual keyboard 212. When the degree of the virtual keyboard 212 is set between “0%” and “100%” (excluding 0% and 100%), the virtual keyboard 212 and the button group 222 are displayed together and the button group 222 can be viewed through the virtual keyboard 212. In this state, the sensor 132 senses a touch operation to the virtual keyboard 212 as a valid key input to the virtual keyboard 212. In this way, an input using the virtual keyboard 212 is performed. In other words, the sensor 132 senses the touch operation to the overlapping portion of the virtual keyboard 212 and the button group 222 not as a valid input to the button group 222 but as a valid input to the virtual keyboard 212 which is displayed in front.

As the degree of the transparency of the virtual keyboard 212 is increased further to be set at 100% (maximum degree of transparency), the virtual keyboard 212 becomes completely transparent. In this case, as shown in FIG. 14C, the virtual keyboard is not visible (for the sake of convenience of description, FIG. 14C shows the virtual keyboard 212 by a broken line). In the overlapping portion, the button group 222 alone is displayed as a background image. In this case, the sensor 132 does not sense a touch operation in the area in which the virtual keyboard 212 is disposed as a valid key input to the virtual keyboard 212, but senses the input as a valid input to the button group 222 on the background. In this way, an input to the button group 222 is sensed as a valid input, even when the layer 210 including the virtual keyboard 212 is disposed in front of the layer 220 including the button group 222.

As shown in FIG. 8, the layer 210 including the virtual keyboard 212 is disposed in front of the layer 220 including the button group 222. Thus, without invalidating the input to the virtual keyboard 212, a touch operation to the button group 222 would be sensed as a valid key input to the virtual keyboard 212 even when the virtual keyboard 212 is completely transparent and the button group 222 alone is displayed in the overlapping portion. When the virtual keyboard 212 is invisible while the button group 222 is visible, it can be assumed, based on a reasonable assumption of the user's intention, that a touch operation to the button group 222 (overlapping portion) is intended to an input operation to the button group 222. If the touch operation to the button group 222 were sensed as a valid input to the virtual keyboard 212, an erroneous input to the keyboard would be caused. In contrast, according to the third display example, the touch operation to the button group 222 is sensed not as a key input to the virtual keyboard 212 but as a valid input to the button group 222. In this way, an erroneous input to the keyboard can be prevented and a proper input to the button group 222 is enabled.

Further, even with the degree of transparency of the virtual keyboard 212 set to a value other than “100%”, the sensor 132 may sense the touch operation not as a valid key input to the virtual keyboard 212 but as a valid input to the button group 222 when the degree of transparency of the virtual keyboard 212 is equal to or higher than a reference value. The reference value is, for example, a predetermined value and can be varied by a user.

Fourth Display Example

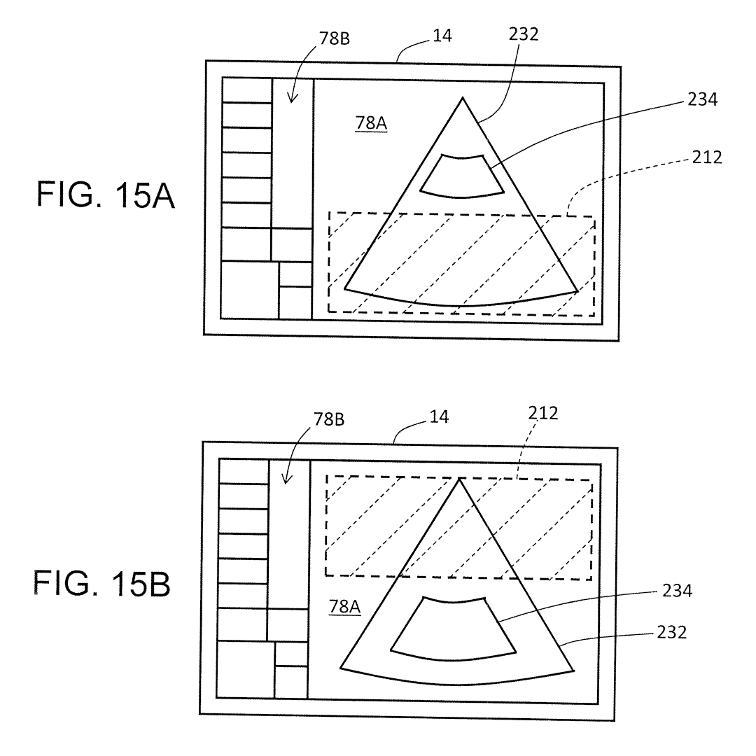

Referring to FIGS. 15A and 15B, the fourth display example of a virtual keyboard is described. In the fourth display example, the display position of the virtual keyboard is changed depending on the position of a point of interest which is set for an ultrasound image (for example, a sample volume used in a Doppler measurement) and the position of a region of interest (ROI).

When a user provides instructions to set a region of interest, the display controller 130 displays a region of interest 234 on the ultrasound image 232, for example, as shown in FIG. 15A. The display position, shape, and size of the region of interest 234 are designated by, for example, a user. In the example shown in FIG. 15A, the region of interest 234 is displayed in the upper area in the display area 78A. In this case, the display controller 130 displays the virtual keyboard 212 in the lower area in the display area 78A. When the region of interest 234 is displayed in the lower area in the display area 78A in accordance with instructions from the user, the display controller 130 displays the virtual keyboard 212 in the upper area in the display area 78A. As such, the vertical position of the virtual keyboard 212 is selected in accordance with the vertical position (in the height direction) of the region of interest 234. In a case where a sample volume is set on the ultrasound image 232, the vertical position of the virtual keyboard 212 is selected in accordance with the vertical position of the sample volume.

In accordance with the fourth display example, the virtual keyboard 212 is displayed by automatically avoiding the display position of the region of interest or the sample volume. In this way, the region of interest and the sample volume become more visible for the user, and the settings of these elements are facilitated.

It should be noted that the display controller 130 may display the virtual keyboard 212 in an intermediate area between the upper area and the lower area by avoiding the display position of the region of interest and the sample volume, or at any position in the vertical direction (height direction).

Other Configuration Examples for Display Control

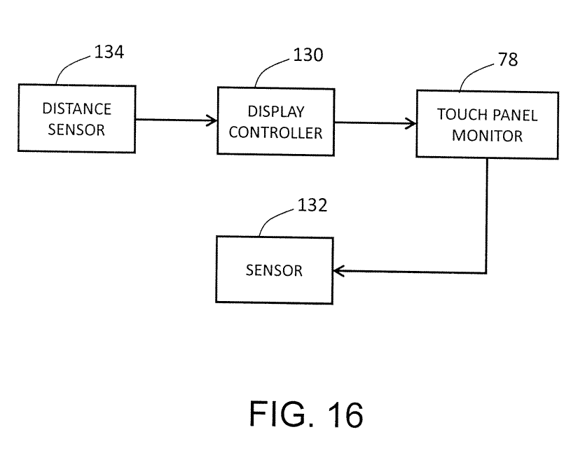

Referring to FIG. 16, another configuration for display control of a touch panel monitor is described. In this example, a distance sensor 134 is used. The distance sensor 134 is disposed, for example, in the vicinity of the touch panel monitor 78 of the BE device 14 for sensing the distance between the touch panel monitor 78 and the user (observer). The sensed values are output to the display controller 130. For the distance sensor 134, a sensor such as an optical sensor, an ultrasound sensor, and a magnetic sensor, may be used.

The display controller 130 changes the degree of transparency of the virtual keyboard 212 in accordance with the sensed value (the distance between the touch panel monitor 78 and the user) from the distance sensor 134. For example, the display controller 130 sets a lower degree of transparency of the virtual keyboard 212 for a shorter distance between the virtual keyboard 212 and the user. In this way, the visibility of the virtual keyboard 212 can be enhanced. Conversely, the display controller 130 sets a higher degree of transparency of the virtual keyboard 212 for a longer distance between the touch panel monitor 78 and the user. In this way, the visibility of the ultrasound image can be enhanced. Specifically, the degree of transparency decreases the closer the user is to the touch panel monitor 78, whereas the degree of transparency increases the farther away the user is from the touch panel monitor 78. For example, the display controller 130 may change the degree of transparency of the virtual keyboard 212 stepwise in accordance with the distance between the touch panel monitor 78 and the user (for example, the degrees of transparency are divided into two or more groups and switched stepwise). Alternatively, the display controller 130 may set the degree of transparency of the virtual keyboard 212 to a first degree of transparency when the distance between the touch panel monitor 78 and the user is equal to or below a predetermined value, and set the degree of transparency to a second degree of transparency, which is larger than the first degree of transparency, when the distance is above the predetermined value. In accordance with the distance, two degrees of transparency can be switched in this manner, or three or more degrees of transparency can be switched stepwise.

As described above, display control depending on user (observer) status becomes possible by changing the degree of transparency of the virtual keyboard 212 in accordance with the distance between the touch panel monitor 78 and the user. In other words, because the user status is assumed in accordance with the distance between the touch panel monitor 78 and the user, the display control depending on the user status becomes possible.

In yet another example, when an error message such as battery low is displayed, the display controller 130 may place a higher priority to display the error message than the virtual keyboard 212. In this case, even when the virtual keyboard 212 is displayed on the touch panel monitor 78, the display controller 130 displays the error message in front of the virtual keyboard 212. When the display position of the virtual keyboard 212 and the display position of the error message are overlapped, the error message is displayed over the virtual keyboard 212. Further, unless an operation to hide the error message is performed, the sensor 132 may not sense the touch operation to the virtual keyboard 212 as a valid key input.

### REFERENCE NUMERALS

10 ultrasound diagnostic system, 12 FE device, 14 BE device, 78 touch panel monitor, 130 display controller, 132 sensor, 134 distance sensor, 212 virtual keyboard, 222 button group, and 232 ultrasound image.

1. An ultrasound diagnostic device comprising:
a display that displays an ultrasound image formed based on a reception signal obtained by transmitting and receiving ultrasound waves;
a display controller that performs control to display a virtual keyboard which can be transparently displayed on a display screen on which the ultrasound image is displayed;
a sensor that senses an input to the virtual keyboard on the display screen; and
a distance sensor that senses a distance between the display and a user,
wherein the display screen includes an operation area and an examination screen area;
the display controller displays,
in the operation area, a button group for inputting a command related to an ultrasound diagnosis,
in the examination screen area, an ultrasound image and the virtual keyboard that is an image different from the button group, three or more degrees of transparency of the virtual keyboard being switched in a stepwise manner in accordance with the distance sensed by the distance sensor; and
the virtual keyboard comprises a plurality of keys for inputting text, the virtual keyboard being used for inputting text.

2. The ultrasound diagnostic device according to claim 1, wherein
the degrees of transparency of the virtual keyboard are variable.

3. The ultrasound diagnostic device according to claim 1, wherein
the display controller shifts the virtual keyboard in an up direction or a down direction within the display screen in accordance with a shift instruction.

4. The ultrasound diagnostic device according to claim 3, wherein
the display controller displays, on the display, a button image for shifting the virtual keyboard in the up direction or the down direction.

5. The ultrasound diagnostic device according to claim 1, wherein
when the degree of transparency of the virtual keyboard satisfies a predetermined condition, the sensor senses an input within a display area of the virtual keyboard as an input to a background image behind the virtual keyboard.

6. The ultrasound diagnostic device according to claim 1, wherein
the display controller increases the degree of transparency of the virtual keyboard when the distance between the display and the user is larger, compared to the degree of transparency when the distance between the display and the user is smaller.

7. The ultrasound diagnostic device according to claim 1, wherein
the display controller changes a display position of the virtual keyboard in accordance with a position of a point of interest or a position of a region of interest, which is set for the ultrasound image.

8. A method for displaying an ultrasound image, comprising:
displaying, on a display, an ultrasound image formed based on a reception signal obtained by transmitting and receiving ultrasound waves and performing control to display a virtual keyboard which can be transparently displayed on a display screen on which the ultrasound image is displayed; and
sensing an input to the virtual keyboard on the display screen,
wherein the display screen includes an operation area and an examination screen area;
in performing the control,
a button group for inputting a command related to an ultrasound diagnosis is displayed in the operation area, and
an ultrasound image and the virtual keyboard that is an image different from the button group are displayed in the examination screen area, three or more degrees of transparency of the virtual keyboard being switched in a stepwise manner in accordance with a distance sensed by a distance sensor that senses a distance between the display and a user;
the virtual keyboard comprises a plurality of keys for inputting text, the virtual keyboard being used for inputting text.

9. (canceled)
